SEQUENCE LISTING (24 sequences)

SEQ ID NO: 1 - protein, 14 aa, synthetic construct
MCMPCFTTDHQMAR

SEQ ID NO: 2 - protein, 116 aa, synthetic construct
EVQLVESGGGLVQPGKSLKLSCEASGFTFSGYGMHWVRQAPGRGLESVAYITSSSINIKYADAVKGRFTVSRDNAKNLLFLQMNILKSEDTAMYYCARFDWDKNYWGQGTMVTVSS

SEQ ID NO: 3 - protein, 256 aa, synthetic construct
EVQLVESGGGLVQPGKSLKLSCEASGFTFSGYGMHWVRQAPGRGLESVAYITSSSINIKYADAVKGRFTVSRDNAKNLLFLQMNILKSEDTAMYYCARFDWDKNYWGQGTMVTVSSGGGGSGGGGSGGGGSQMTQSPSSLPASLGDRVTINCQASQDISNYLNWYQQKPGKAPKLLIYYTNKLADGVPSRFSGSGSGRDSSFTISSLESEDIGSYYCQQYYNYPWTFGPGTKLEIKRGGGGSMCMPCFTTDHQMAR

SEQ ID NO: 4 - protein, 256 aa, synthetic construct; identical to SEQ ID NO: 3

SEQ ID NO: 5 - protein, 264 aa, synthetic construct
HHHHHHHHEVQLVESGGGLVQPGKSLKLSCEASGFTFSGYGMHWVRQAPGRGLESVAYITSSSINIKYADAVKGRFTVSRDNAKNLLFLQMNILKSEDTAMYYCARFDWDKNYWGQGTMVTVSSGGGGSGGGGSGGGGSQMTQSPSSLPASLGDRVTINCQASQDISNYLNWYQQKPGKAPKLLIYYTNKLADGVPSRFSGSGSGRDSSFTISSLESEDIGSYYCQQYYNYPWTFGPGTKLEIKRGGGGSMCMPCFTTDHQMAR

SEQ ID NO: 6 - DNA, 6460 bp, synthetic construct
  source 1..6460: mol_type = other DNA
ttctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatccggatatagttcctcctttcagcaaaaaacccctcaagacccgtttagaggccccaaggggttatgctagttattgctcagcggtggcagcagccaactcagcttcctttcgggctttgttagcagccggatcctcaacgtgccatctggtgatcagtagtgaagcacggcatacacatggagccaccaccaccacgcttgatttccagcttagtacccggcccgaaagtccacgggtagttatagtactgctggcagtaataagagccgatatcttcggattcgagggaggagatagtgaaagaagaatcacggccagaaccagaaccgctgaaacgggacggtaccccatctgccagtttgttagtgtagtagatcagcagcttcggagccttacccggcttctgctggtaccagttcagatagttgctgatgtcctggctagcctgacagttgatagtaacacggtcgcccagggatgcaggcagggaagacggagactgagtcatctgggagccaccaccaccggacccaccgccaccggagccaccgccaccggaggatacagttaccatagtgccctggccccagtagttcttgtcccaatcgaaacgagcgcagtaatacattgcagtgtcttcagacttcaggatgttcatctgcaggaacagcaggttctttgcgttgtcacgggatacagtgaaacgaccttttacagcgtcagcgtacttgatgttgatgctggagctagtgatatatgctacgctttccaggccacgacccggagcctgacgtacccagtgcatgccgtacccgctgaaagtgaagccagatgcttcacaggacagtttcagggatttgcccggctgaaccagaccgccaccagattcaaccagctgaacttctccctggaagtacaggttttcatggtgatggtggtgatggtggtgcatatgtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 7 - protein, 36 aa, Leiurus quinquestriatus
MCMPCFTTDHQMARKCDDCCGGKGRGKCYGPQCLCR

SEQ ID NO: 8 - protein, 36 aa, Buthus eupeus
MCMPCFTTRPDMAQQCRACCKGRGKCFGPQCLCGYD

SEQ ID NO: 9 - protein, 36 aa, Buthus eupeus
MCMPCFTTDHQTARRCRDCCGGRGRKCFGQCLCGYD

SEQ ID NO: 10 - protein, 35 aa, Buthus eupeus
MCMPCFTTDHNMAKKCRDCCGGNGKCFGPQCLCNR

SEQ ID NO: 11 - protein, 35 aa, Buthus eupeus
MCMPCFTTDPNMANKCRDCCGGGKKCFGPQCLCNR

SEQ ID NO: 12 - protein, 35 aa, Buthus eupeus
MCMPCFTTDPNMAKKCRDCCGGNGKCFGPQCLCNR

SEQ ID NO: 13 - protein, 35 aa, Buthus sindicus
RCKPCFTTDPQMSKKCADCCGGKGKGKCYGPQCLC

SEQ ID NO: 14 - protein, 38 aa, Leiurus hebraeus
RCSPCFTTDQQMTKKCYDCCGGKGKGKCYGPQCICAPY

SEQ ID NO: 15 - protein, 25 aa, Parabuthus schlechteri
RCKPCFTTDPQMSKKCADXCGGXKX

SEQ ID NO: 16 - protein, 36 aa, Buthus sindicus
CGPCFTKDPETEKKCATCCGGIGRCFGPQCLCNRGY

SEQ ID NO: 17 - protein, 35 aa, Androctonus mauretanicus
CGPCFTTDPYTESKCATCCGGRGKCVGPQCLCNRI

SEQ ID NO: 18 - protein, 34 aa, Androctonus australis
MCIPCFTTNPNMAAKCNACCGSRRGSCRGPQCIC

SEQ ID NO: 19 - protein, 34 aa, Leiurus hebraeus
CGPCFTTDHQMEQKCAECCGGIGKCYGPQCLCNR

SEQ ID NO: 20 - protein, 35 aa, Buthus martensii
CGPCFTTDANMARKCRECCGGIGKCFGPQCLCNRI

SEQ ID NO: 21 - protein, 35 aa, Buthus martensii
CGPCFTTDANMARKCRECCGGNGKCFGPQCLCNRE

SEQ ID NO: 22 - protein, 37 aa, Buthus tamulus
RCGPCFTTDPQTQAKCSECCGRKGGVCKGPQCICGIQ

SEQ ID NO: 23 - protein, 35 aa, Buthus tamulus
RCPPCFTTNPNMEADCRKCCGGRGYCASYQCICPG

SEQ ID NO: 24 - protein, 29 aa, Leiurus hebraeus
VSCEDCPDHCSTQKARAKCDNDKCVCEPI

(19) United States
(12) Patent Application Publication
(10) Pub. No.: US 2023/0382960 A1
(43) Pub. Date: Nov. 30, 2023

(54) TRUNCATED CHLOROTOXIN FUSION PROTEINS AND METHODS OF USE THEREOF

(71) Applicants: Tsafrir S. LEKET-MOR, Tempe, AZ (US); Braeden SCHAEFER, Phoenix, AZ (US); Rebecca COOK, Mesa, AZ (US); Joseph BLATTMAN, Scottsdale, AZ (US)

(72) Inventors: Tsafrir S. LEKET-MOR, Tempe, AZ (US); Braeden SCHAEFER, Phoenix, AZ (US); Rebecca COOK, Mesa, AZ (US); Joseph BLATTMAN, Scottsdale, AZ (US)

(73) Assignee: Arizona Board of Regents on Behalf of Arizona State University, Scottsdale, AZ (US)

(21) Appl. No.: 18/324,102

(22) Filed: May 25, 2023

Related U.S. Application Data
(60) Provisional application No. 63/346,770, filed on May 27, 2022.

(51) Int. Cl. C07K 14/435; C07K 16/28; A61P 35/00

(52) U.S. Cl. CPC C07K 14/43522; C07K 16/2809; A61P 35/00; C07K 2319/00; A61K 38/00

(57) ABSTRACT

The present invention relates to a truncated form of chlorotoxin and chlorotoxin-like protein that are useful for glioma immunotherapy, in particular the treatment of glioblastoma. Thus, the invention also encompasses a therapeutic fusion protein comprising the truncated form of chlorotoxin and chlorotoxin-like protein, its methods of use, and its method of production.

### CROSS-REFERENCE TO RELATED APPLICATIONS

This application claims the benefit of U.S. Provisional Application No. 63/346,770, entitled “Truncated Chlorotoxin Fusion Proteins and Methods of Use Thereof,” which was filed May 27, 2022, the entire disclosure of which is hereby incorporated herein by this reference.

### INCORPORATION-BY-REFERENCE OF MATERIAL ELECTRONICALLY FILED

Incorporated by reference in its entirety herein is a computer-readable nucleotide/amino acid sequence listing submitted concurrently herewith and identified as follows: One 29,164 byte XML file named “SeqList” created on May 22, 2023.

### FIELD OF THE INVENTION

The invention relates to novel fusion proteins for use in cancer immunotherapy, in particular the treatment of glioblastoma.

### BACKGROUND OF THE INVENTION

Glioblastoma multiforme (GBM) is the most common and aggressive primary brain tumor and the second most common cancer of the central nervous system (CNS), comprising 15% of all intracranial neoplasms and 60-75% of tumors of astrocytic origin. GBM can arise as either a primary or secondary tumor, both of which exhibit high heterogeneity. Primary GBM typically occurs spontaneously as a grade IV astrocytoma in the white matter of the brain and can grow rapidly before patients experience symptoms, leading to late detection of the tumor and a less favorable prognosis. Conversely, secondary GBM tumors arise from lower-grade astrocytomas (grades I-III) and grow much slower, leading to a slight increase in post-detection survival time. While GBM arises and metastasizes primarily within the brain, reports of tumor spread to the spinal cord through the cerebrospinal fluid (CSF) have been recorded, though these instances are very rare.

Minimal progress has been made regarding treatment strategies for GBM over the past three decades. The current standard of care—a combination of surgical resection, chemotherapy, and radiotherapy—is employed more so to prolong patient survival rather than to eliminate the tumor entirely, as even in cases of maximum treatment, GBM typically recurs. While these treatments do increase median survival from 3 months to 12-15 months following diagnosis, 5-year survival remains a dismal 3-7%. The aggressively metastatic nature of GBM in combination with its current, inadequate treatment strategy leads to tumor recurrence, and eventually death, in nearly 100% of patients.

The poor prognosis of GBM is aided by numerous obstacles that stand in the way of its treatment, obstacles that the current standard of care often cannot overcome. These include but are not limited to: (1) metastatic infiltration of GBM within the brain, (2) protection from the blood-brain barrier (BBB), and (3) immune evasion tactics employed within the tumor microenvironment. Identifying and understanding these barriers to treatment in the context of the current care model provides useful insight that can be used to inform the development of more successful therapies for GBM going forward.

The foremost barrier to treatment of GBM involves the tumor's aggressive, metastatic nature. Infiltration of GBM cells throughout the brain gives rise to complications pertaining to complete surgical removal of the tumor, as small populations of malignant cells capable of forming recurrent, satellite tumors often go undetected and thus are not removed during surgery. Surgery is likewise complicated by the location of the tumor, where complete resection of the tumor without removing healthy brain tissue becomes a difficult and dangerous task. As a result, surgical resection cannot be relied upon for complete removal of the tumor, and especially for satellite tumors that may form from small populations of infiltrative cells throughout the brain. Elimination of the complete tumor without harming healthy brain tissue is also a cause of concern for treatment via chemotherapy and radiotherapy, which are typically employed following initial resection to attempt to eliminate any remaining cell populations. While these treatments can slow recurrent tumor progression, the heterogeneity of GBM and the presence of cancer stem cells that up-regulate responses to DNA damage often result in resistance against these therapies. Due to the high rate of GBM recurrence and the ability of small populations of infiltrative cells to give rise to new satellite tumors, it is pertinent that any effective treatment is capable of eliminating 100% of GBM cells but not healthy tissue.

Another obstacle standing in the way of GBM treatment lies in its protection provided by the blood-brain barrier, a filtering mechanism of brain vasculature that prevents the passage of certain potentially harmful molecules, and often therapeutic drugs, into the brain. Of these excluded molecules, >98% of chemotherapeutics are denied entry, leading to questions regarding the efficacy of chemotherapy in the context of GBM treatment. The most convincing case for chemotherapy thus far showed that combined treatment with radiation therapy increased the median survival of patients versus those receiving radiation alone; however, this resulted in an increase of only 2.5 months (14.6 vs. 12.1 months, respectively). These observations inform yet another necessary element of an effective therapeutic designed for GBM: it must be able to cross the BBB.

The final barrier to treating GBM discussed here involves its ability to evade the host immune response and induce immunosuppression within the tumor microenvironment.

Specifically, GBM prevents antigen-presenting cells (APCs) from activating the anti-tumor immune response by blocking their transport to the lymph nodes through the secretion of cytokines TGF-β and IL-10. These cytokines also act to induce T regulatory cells (Tregs) and suppress T cell activation, respectively. Induction of Tregs aids in immune evasion by inhibiting and causing exhaustion of cytotoxic CD8+T cells, adding to the limitation of anti-tumor activity by the immune system. GBM is also known to down-regulate expression of MHC Class I and II receptors on the cell surface, preventing recognition from CD8+T cells which would normally initiate perforin-granzyme mediated apoptosis against the tumor. Current treatment strategies also contribute to GBM's ability to evade the immune response. Temozolomide (TMZ), the only chemotherapeutic drug currently approved for treatment of GBM, is known to induce immunosuppression in patients at the doses currently utilized. Combined, these efforts result in an overall reduction in immune-mediated cytotoxic activity against GBM and strongly suggest that an effective treatment should be capable of activating an anti-tumor immune response.

The growing field of immunotherapy stands as one of the most promising alternatives to the current standard of care for GBM. Intended to utilize and stimulate the immune system against disease, emerging immunotherapeutic strategies offer the specificity required to target and eliminate GBM in ways that the current standard of care cannot. This specificity allows for the targeting and elimination of populations of cells that may survive first-line treatments, thereby preventing infiltration and formation of satellite tumors. In addition, these therapies aim to prevent off-target toxicity associated with current treatment options by targeting only the cancer cells of interest and not healthy tissue. Recent attempts at designing immunotherapies for GBM have been encouraging but largely disappointing, due in part to the lack of known universal targets for GBM and the tumor's heterogeneity. The most notable target of attempted GBM immunotherapies is a mutation of the epidermal growth factor receptor (EGFR) known as EGFRvIII, which is expressed by >75% of GBM cells in 30% of patients. This variant was the target of an early peptide vaccine designed to initiate an immune response against the tumor through the creation of antibodies against the mutated receptor. Despite a marked increase in median survival among patients in early stages of clinical trials, the vaccine ultimately failed; biopsies of patients also showed decreased expression of EGFRvIII, suggesting the potential for treatment resistance.

Thus, there remains a great need for novel immunotherapy strategies to target GBM cells.

### SUMMARY OF THE INVENTION

Described herein are a truncated form of chlorotoxin or chlorotoxin-like protein that retains native protein's ability to specifically bind to glioma cells, in particular glioblastoma cells. In certain embodiments, the truncated form is a polypeptide of 13-18 amino acid residues comprising a sequence with at least 75% identity to SEQ ID NO:1. In some aspects, the polypeptide of 13-18 amino acid residues comprises one cysteine residue, two cysteine residues, or three cysteine residues. The cysteine residues are conserved residues from chlorotoxin or chlorotoxin-like protein. In certain embodiments, the polypeptide of 13-18 amino acid residues specifically binds to glioblastoma cells. In a particular embodiment, the amino acid sequence of the truncated form comprises SEQ ID NO:1.

Also described herein are therapeutic fusion proteins with the truncated form of chlorotoxin. In some aspects, the therapeutic fusion protein comprises a truncated form of chlorotoxin having 13-18 amino acid residues in length and comprising a sequence with at least 75% homology to SEQ ID NO:1; a VH domain of an antibody against CD3; and a VL domain of an antibody against CD3. A first amino acid linker sequence links the C-terminus of the VH domain the antibody against CD3 to the N-terminus of VL domains of the antibody against CD3. A second amino acid linker sequence links the C-terminus of the truncated form of chlorotoxin to the N-terminus of the VH domain of an antibody against CD3. In some embodiments, the first amino acid linker and the second amino acid linkers consist of at least one glycine residue and at least one serine residue. In certain embodiments, the amino acid sequence of the therapeutic fusion protein comprises the sequence set forth in SEQ ID NO:4. Also described herein is a plasmid for expressing therapeutic fusion protein. In some embodiments, the sequence of the plasmid comprises SEQ ID NO:6.

A method of producing a glioma-targeting therapeutic fusion protein is also described. The method comprises linking an anti-glioma therapeutic agent to the truncated form of chlorotoxin or chlorotoxin-like protein to produce the glioma-targeting therapeutic fusion protein. In some aspects, the C-terminus of the polypeptide is linked to the anti-glioma therapeutic agent, for example, by an amino acid linker sequence. In certain implementations, the amino acid linker sequence consists of at least one glycine residue and at least one serine residue. In particular embodiments, the anti-glioma therapeutic agent comprises a VH domain and a VL domain of an antibody against CD3. In some aspects, the amino acid sequence of the fusion protein comprises the sequence set forth in SEQ ID NO:4.

A method of treating a subject having glioma is further described herein. The method comprises administering to the subject the glioma-targeting therapeutic fusion protein described above. In some implementations, the glioma-targeting therapeutic fusion protein a truncated form of chlorotoxin being 13-18 amino acid residues comprising a sequence with at least 75% homology to SEQ ID NO:1 and an anti-glioma therapeutic agent. In certain implementations, the truncated form of chlorotoxin consists of the sequence set forth in SEQ ID NO:1. In some aspects, the anti-glioma therapeutic agent comprises a V_{H} domain and a V_{L} domain of an antibody against CD3. In a particular implementation, the subject is administered a fusion protein with an amino acid sequence comprising the sequence set forth in SEQ ID NO:4 or SEQ ID NO:5.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A and 1B depict, in accordance with certain embodiments, the design of the therapeutic fusion protein (FIG. 1A) and its purported interaction with glioblastoma cells (FIG. 1B).

FIGS. 2A-2C depict, in accordance with certain embodiments, the design and expression of ACDClxΔ15.

FIGS. 3A-3D depict, in accordance with certain embodiments, analysis of GBM binding via immunocytochemistry.

FIGS. 4A-4D depict, in accordance with certain embodiments, analysis of ACDClxΔ15-GMB binding via flow cytometry.

FIGS. 5A-5C depict, in accordance with certain embodiments, analysis of GBM-T-cell interactions via flow cytometry and FACS.

### DETAILED DESCRIPTION OF THE INVENTION

Detailed aspects and applications of the invention are described below in the drawings and detailed description of the invention. Unless specifically noted, it is intended that the words and phrases in the specification and the claims be given their plain, ordinary, and accustomed meaning to those of ordinary skill in the applicable arts.

In the following description, and for the purposes of explanation, numerous specific details are set forth in order to provide a thorough understanding of the various aspects of the invention. It will be understood, however, by those skilled in the relevant arts, that the present invention may be practiced without these specific details. It should be noted that there are many different and alternative configurations, devices, and technologies to which the disclosed inventions may be applied. The full scope of the inventions is not limited to the examples that are described below.

The singular forms “a,” “an,” and “the” include plural referents unless the context clearly dictates otherwise. Thus, for example, reference to “a step” includes reference to one or more of such steps.

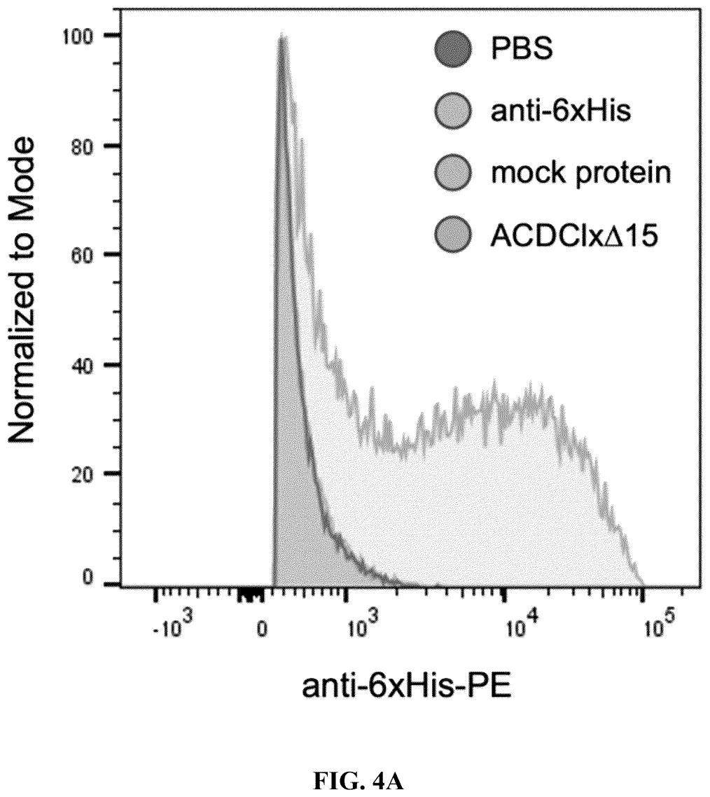

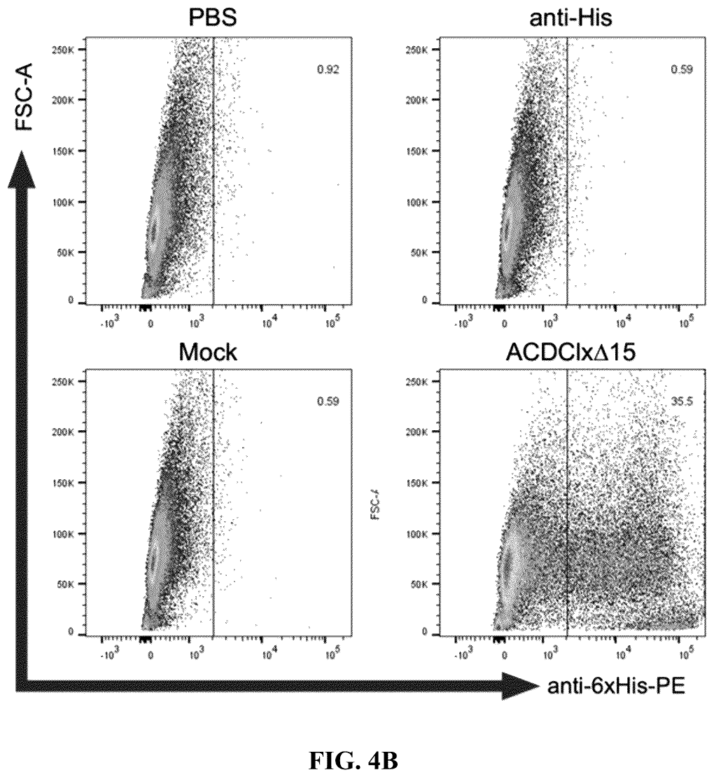

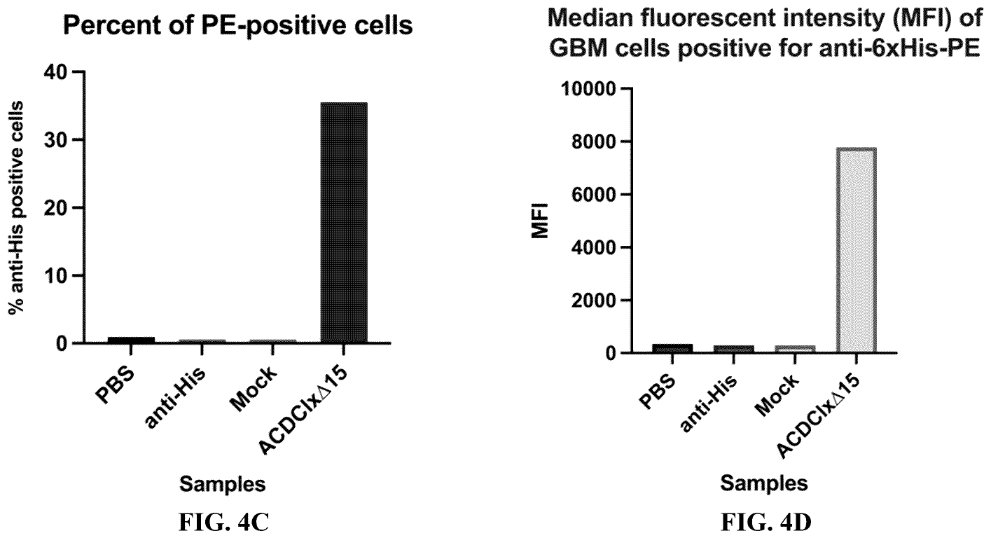

As used herein, the term “chlorotoxin-like peptide” refers to a peptide with at least 60% sequence identity to chlorotoxin (UniProt KB P45639.1) comprising at least three di-sulfide bridges. A list of representative chlorotoxin-like peptides may be found in Table 1, reproduced from Dardevet et al, Toxins (Basel). 2015, 7(4): 1079-1101, and in FIG. 4 of Ali et al., Toxins. 2016, 8(2):36. The contents of Dardevet et al., and Ali et al., are incorporated by reference herein.

TABLE 1
Primary sequence alignments of chlorotoxin-like protein. Alignments were
performed by using @TOME V2. Percentage sequence of identity is given as compared to
chlorotoxin by using @TOME V2. Disulfide Bridge pattern is given when known. Cysteine
residues involved in disulfide bridges appear in blue in the table and are numbered in
order of appearance.

| | | | | Disulfide | |
|---|---|---|---|---|---|
| | | | | bridge | |
| Toxin | Primary sequence | Length | Identity | pattern | Species |
| Chlorotoxin | MC₁MPC₂FTTDHQMARKC₃DDC₄C₅G- | 36 AA | 100% | C₁-C₄, | Leiurus |
| | GK-GRGKC₆YGPQC₇LC₈-R | | | C₂-C₆, | quinquestriatus |
| | (SEQ ID NO. 7) | | | C₃-C₇, | quinquestriatus |
| | | | | C₅-C₈ | |
| I₁ | MC₁MPC₂FTTRPDMAQQC₃RAC₄C₅K- | 36 AA | 71% | C₁-C₄, | Buthus eupeus |
| | GR-GK--C₆FGPQC₇LC₈GYD- | | | C₂-C₆, | |
| | (SEQ ID NO. 8) | | | C₃-C₇, | |
| | | | | C₅-C₈ | |
| I₃ | MC₁MPC₂FTTDHQTARRC₃RDC₄C₅G- | 36 AA | 82% | C₁-C₄, | Buthus eupeus |
| | GR-GR-KC₆FG-QC₇LC₈GYD- | | | C₂-C₆, | |
| | (SEQ ID NO. 9) | | | C₃-C₇, | |
| | | | | C₅-C₈ | |
| I₄ | MC₁MPC₂FTTDHNMAKKC₃RDC₄C₅G- | 35 AA | 82% | C₁-C₄, | Buthus eupeus |
| | GN---GKC₆FGPQC₇LC₈NR | | | C₂-C₆, | |
| | (SEQ ID NO. 10) | | | C₃-C₇, | |
| | | | | C₅-C₈ | |
| I₅ | MC₁MPC₂FTTDPNMANKC₃RDC₄C₅G- | 35 AA | 79% | C₁-C₄, | Buthus eupeus |
| | GG-KK--C₆FGPQC₇LC₈NR- | | | C₂-C₆, | |
| | (SEQ ID NO. 11) | | | C₃-C₇, | |
| | | | | C₅-C₈ | |
| I_{5A} | MC₁MPC₂FTTDPNMAKKC₃RDC₄C₅G- | 35 AA | 79% | C₁-C₄, | Buthus eupeus |
| | GN-GK--C₆FGPQC₇LC₈NR- | | | C₂-C₆, | |
| | (SEQ ID NO. 12) | | | C₃-C₇, | |
| | | | | C₅-C₈ | |
| Bs-8 | RC₁KPC₂FTTDPQMSKKC₃ADC₄C₅G-GK- | 35 AA | 80% | C₁-C₄, | Buthus |
| | GKGKC₆YGPQC₇LC₈---- | | | C₂-C₆, | sindicus |
| | (SEQ ID NO. 13) | | | C₃-C₇, | |
| | | | | C₅-C₈ | |
| Lqh-8/6 | RC₁SPC₂FTTDQQMTKKC₃YDC₄C₅G-GK- | 38 AA | 72% | C₁-C₄, | Leiurus |
| | GKGKC₆YGPQC₇IC₈APY- | | | C₂-C₆, | quinquestriatus |
| | (SEQ ID NO. 14) | | | C₃-C₇, | hebraeus |
| | | | | C₅-C₈ | |
| PBITx1 | RC₁KPC₂FTTDPQMSKKC₃ADX₄C₅G-GX-- | 25 AA | 64% | | Parabuthus |
| | KX | | | | schlechteri |
| | (SEQ ID NO. 15) | | | | |
| Bs-14 | -C₁GPC₂FTKDPETEKKC₃ATC₄C₅G-GI- | 36 AA | 61% | C₁-C₄, | Buthus |
| | GR--C₆FGPQC₇LC₈NRGY | | | C₂-C₆, | sindicus |
| | (SEQ ID NO. 16) | | | C₃-C₇, | |
| | | | | C₅-C₈ | |
| Neurotoxin P2 | -C₁GPC₂FTTDPYTESKC₃ATC₄C₅G-GR- | 35 AA | 70% | C₁-C₄, | Androctonus |
| | GK--C₆VGPQC₇LC₈NRI- | | | C₂-C₆, | mauretanicus |
| | (SEQ ID NO. 17) | | | C₃-C₇, | mauretanicus |
| | | | | C₅-C₈ | |
| AaCtx | MC₁IPC₂FTTNPNMAAKC₃NAC₄C₅G- | 34 AA | 61% | C₁-C₄, | Androctonus |
| | SRRGS--C₆RGPQC₇IC₈---- | | | C₂-C₆, | australis |
| | (SEQ ID NO. 18) | | | C₃-C₇, | |
| | | | | C₅-C₈ | |
| GaTx1 | -C₁GPC₂FTTDHQMEQKC₃AEC₄C₅G-GI- | 34 AA | 79% | C₁-C₄, | Leiurus |
| | GK--C₆YGPQC₇LC₈NR---- | | | C₂-C₆, | quinquestriatus |
| | (SEQ ID NO. 19) | | | C₃-C₇, | hebraeus |
| | | | | C₅-C₈ | |
| BmKCT | -C₁GPC₂FTTDANMARKC₃REC₄C₅G-GI- | 35 AA | 76% | C₁-C₄, | Buthus |
| | GK--C₆FGPQC₇LC₈NRI- | | | C₂-C₆, | martensii |
| | (SEQ ID NO. 20) | | | C₃-C₇, | |
| | | | | C₅-C₈ | |
| Bm12-b | -C₁GPC₂FTTDANMARKC₃REC₄C₅G-GN- | 35 AA | 76% | C₁-C₄, | Buthus |
| | GK--C₆FGPQC₇LC₈NRE- | | | C₂-C₆, | martensii |
| | (SEQ ID NO. 21) | | | C₃-C₇, | |
| | | | | C₅-C₈ | |
| Lepidopteran | RC₁GPC₂FTTDPQTQAKC₃SEC₄C₅G-RK- | 37 AA | 63% | C₁-C₄, | Buthus tamulus |
| | GG-VC₆KGPQC₇IC₈GIQ- | | | C₂-C₆, | |
| | (SEQ ID NO. 22) | | | C₃-C₇, | |
| | | | | C₅-C₈ | |
| BtITx3 | RC₁PPC₂FTTNPNMEADC₃RKC₄C₅G-GR-- | 35 AA | 53% | C₁-C₄, | Buthus tamulus |
| | GY-C₆ASYQC₇IC₈PG---- | | | C₂-C₆, | |
| | (SEQ ID NO. 23) | | | C₃-C₇, | |
| | | | | C₅-C₈ | |
| GaTx2 | --VSC₁--------EDC₂PDHC₃STQK- | 29 AA | 38% | C₁-C₄, | Leiurus |
| | ARAKC₄DNDKC₅VC₆-EPI | | | C₂-C₅, | quinquestriatus |
| | (SEQ ID NO. 24) | | | C₃-C₆ | hebraeus |

Immunotherapies such as the bispecific T cell engager represent the most promising of new, experimental treatment strategies for glioblastoma in part due to their unique ability to stimulate T cell responses against cancer. Although capable of surpassing many of the hurdles standing in the way of effective GBM treatment, these immunotherapies have been largely ineffective due to the lack of a universal target for GBM. Chlorotoxin, a scorpion-derived peptide, has been shown to overcome this through its unique ability to bind human glioma cells without off-target toxicity to other tissues.

Disclosed herein is a truncated form of chlorotoxin and chlorotoxin-like protein that are useful for glioma immunotherapy, in particular the treatment of glioblastoma. Nearly 20 years ago, researchers studying chlorotoxin, a 36-amino acid peptide found in the venom of the deathstalker scorpion (L. quinquestriatus), reported its unique ability to selectively bind glioma cells but not healthy tissue. This affinity for glioma increases in proportion to the grade of the tumor, with chlorotoxin binding 100% of grade IV glioma (GBM) cells (31/31 samples positive). Importantly, chlorotoxin also displayed high selectivity for GBM, as it was found to not bind samples of various healthy human tissues. Together, these qualities make the peptide an interesting candidate for use in GBM treatment.

Owing to its selectivity for glioma cells and lack of toxicity to humans, chlorotoxin has found relevance as a supplementary tool within the current standard of care. In the clinical setting, a modified chlorotoxin molecule attached to a Cy5.5 infrared dye, coined Tumor Paint, has been utilized to enhance visualization of the tumor during surgery in mice and is awaiting clinical trials in humans. Despite intensive research and promising clinical applications, chlorotoxin's binding target on glioma cells remains unknown. Multiple potential targets have been reported, including matrix metalloproteinase 2 (MMP-2), annexin A2, Nrp1, and glioma-specific chloride channels, yet none have demonstrated evidence of direct interaction with chlorotoxin. Regardless, it is clear that chlorotoxin possesses incredible therapeutic potential as a targeting molecule for GBM.

Eight of chlorotoxin's 36 amino acids are cysteine residues, which form four disulfide bonds (DSBs) in a highly compact inhibitor-cysteine knot (ISK) motif. These DSBs posed problems during the expression of ACDClx, a chlorotoxin-conjugated immunotherapeutic protein and the subject of U.S. Pat. No. 10,745,478, in both Nicotiana benthamiana and Escherichia coli, as in both systems DSB formation outside of its natural context resulted in largely insoluble, aggregated protein. In E. coli, DSB formation during recombinant expression has been previously achieved by selectively expressing the gene of interest within the periplasm; however, periplasm targeted ACDClx remained insoluble in solution. Recently, researchers studying chlorotoxin as part of a peptide-drug conjugate (PDC) reported that chlorotoxin was metabolized in the GBM tumor microenvironment to form peptide fragments that retained their GBM-binding capabilities. Interestingly, many of these fragments all retained a C-terminal arginine residue thought to be responsible for GBM binding.

For the first time, a truncated form of chlorotoxin comprising 13-18 amino acid residues was found to be sufficient for specific binding to glioma cells, specifically glioblastoma cells. This truncated form of chlorotoxin was selected for minimizing disulfide bonds and thus only comprises the first three cysteines of a chlorotoxin or chlorotoxin-like protein, as the first disulfide bond in chlorotoxin and chlorotoxin-like proteins is formed between the first and fourth cysteine residues. Thus, the polypeptide with specific binding to glioma cells has 13-18 contiguous residues of the amino acid sequence of chlorotoxin and chlorotoxin-like protein. In some aspects, the truncated form comprises the first 13-18 contiguous amino acid residues of chlorotoxin or chlorotoxin-like protein. This deletion reduces the number of cysteine residues present within the peptide from eight to two, thereby allowing for the formation of only one possible DSB within the truncated chlorotoxin molecule. In particular embodiments, the polypeptide with specific binding to glioma cells comprises a sequence with at least 75%, at least 80%, or at least 90% sequence identity to SEQ ID NO:1. In some aspects, such a polypeptide comprises no more than three cysteine residues. Thus, in particular embodiments, the polypeptide of 13-18 amino acid residues comprises one cysteine residue, two cysteine residues, or three cysteine residues.

Accordingly, also described herein is a method of targeting a therapeutic agent to glioma cells. The method comprises linking the therapeutic agent to the above-described polypeptide with specific binding to glioma cells to produce a fusion protein; and administering the fusion protein to a subject, wherein the subject has glioma cells. In some embodiments, the polypeptide with specific binding to glioma cells has an amino acid sequence comprises a sequence with at least 75%, at least 80%, or at least 90% sequence identity to SEQ ID NO:1. The amino acid sequence of the polypeptide preferably comprises less than three cysteine residues. In particular embodiments, the polypeptide with specific binding to glioma cells has an amino acid sequence consisting of SEQ ID NO:1 (also referred to herein as “CltxΔ15”). In some aspects, the C-terminus of the polypeptide with specific binding to glioma cells is linked to the therapeutic agent, for example with an amino acid linker. In certain embodiments, the amino acid linker consists of at least one glycine residue and at least one serine residue. In particular implementations, the method targets the therapeutic agent to glioblastoma cells.

Also described herein is a therapeutic fusion protein comprising the polypeptide with specific binding to glioma cells and a V_{H} domain and a V_{L} domain of an antibody against CD3. This therapeutic fusion protein is designed based on the bispecific T cell engager (BiTE™) model, which is a method originally proposed in 1985 to promote T cell-mediated destruction of target cells. BiTE proteins comprise two single-chain variable fragments (scFvs) of antibodies which are tethered together by a short, inert linker to form a fusion protein. The therapeutic fusion protein described herein comprises the polypeptide with specific binding to glioma cells in place of one of the scFvs, while the other scFvs comprise the V H domain and the V L domain of an antibody against CD3, for example, an antibody against CD3c, where binding leads to structural changes and eventual signal transduction to induce sustained activation of T cells when the target cell is also bound by its complementary antibody. Accordingly, the therapeutic fusion protein further comprises a first amino acid linker sequence that links the C-terminus of the V H domain the antibody against CD3 to the N-terminus of V L domains of the antibody against CD3 and a second amino acid linker sequence that links the C-terminus of the polypeptide with specific binding to glioma cells to the N-terminus of the V_{H} domain of an antibody against CD3. This method of inducing T cell activation is notable in that it does not require recognition by the major histocompatibility complex (MHC), allowing for T cell-mediated elimination of target cells regardless of TCR specificity. Additionally, it overcomes the problem of MHC-downregulation by cells such as GBM which are known to do so in order to evade the immune response.

In some aspects, the V_{H} domain of the antibody against CD3 comprises the sequence set forth in SEQ ID NO:2 or a sequence having at least 50% identity, at least 60% identity, at least 70% identity, at least 80% identity, at least 90% identity, or at least 95% identity to SEQ ID NO:2. In some aspects, the V H domain of the antibody against CD3 comprises the sequence set forth in SEQ ID NO:3 or a sequence having at least 50% identity, at least 60% identity, at least 70% identity, at least 80% identity, at least 90% identity, or at least 95% identity to SEQ ID NO:3. In some embodiments, the first amino acid linker and the second amino acid linkers consist of at least one glycine residue and at least one serine residue. In a particular embodiment, the amino acid sequence of the therapeutic fusion protein comprises the sequence set forth in SEQ ID NO:4. In a certain embodiment, the amino acid sequence of the therapeutic fusion protein is the sequence set forth in SEQ ID NO:5 (also referred to herein as “ACDClxΔ15”).

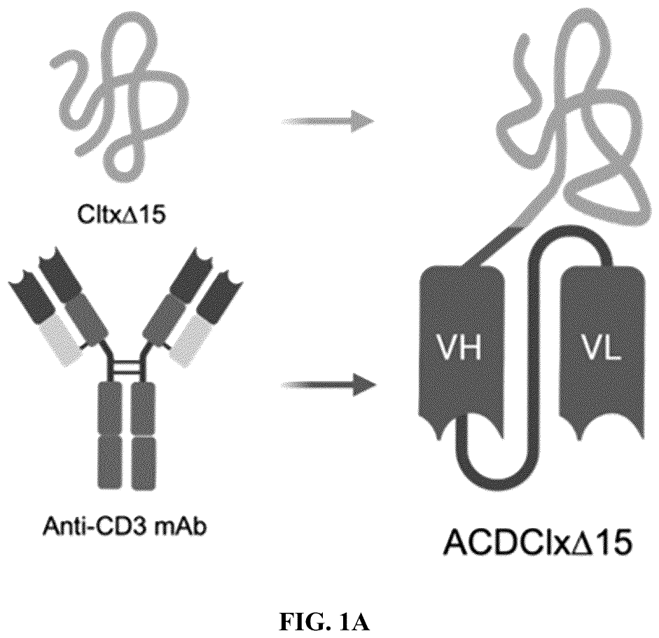

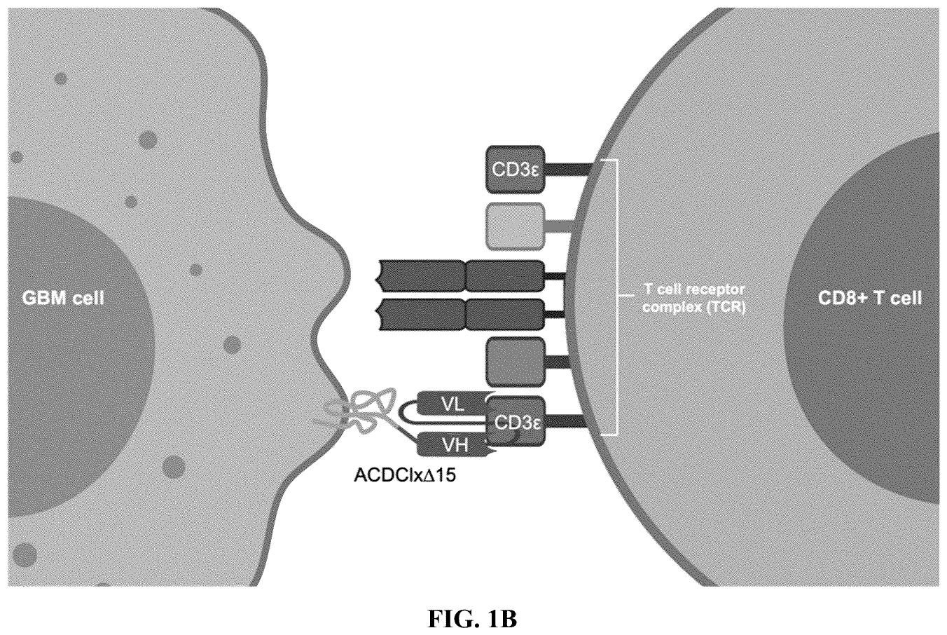

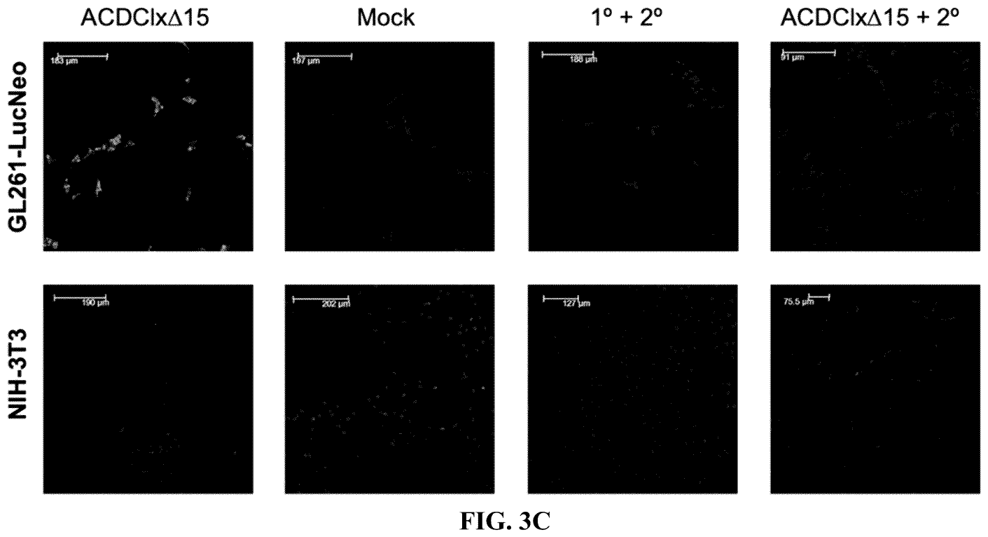

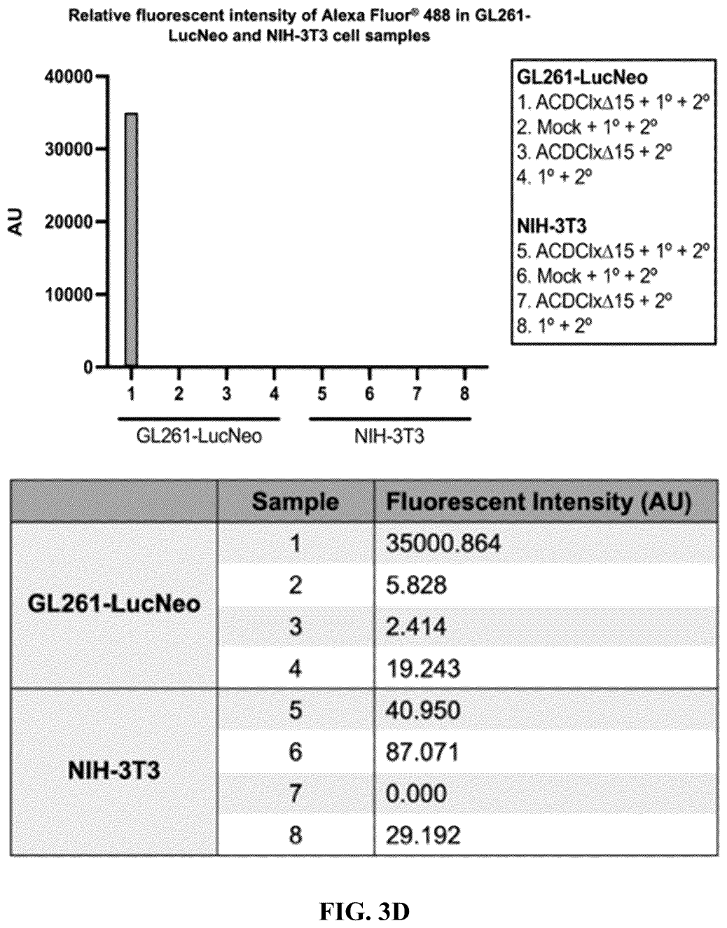

As demonstrated in the Examples, the truncated form of chlorotoxin allow for more efficient production of ACDClxΔ15 while still retaining its ability to bind GBM cells. When GBM cells and NIH-3T3 mouse embryonic fibroblast cells were incubated with ACDClxΔ15, which was then probed for its N-terminal histidine-tag with fluorescent antibodies. Confocal microscopy of these samples revealed that ACDClxΔ15 did bind GBM cells but did not bind healthy fibroblast cells (FIGS. 3A and 3C). These results were verified using flow cytometry to measure the number of cells that were positive for ACDClxΔ15. As expected, ACDClxΔ15 further demonstrated its ability to bind GBM cells in vitro, with high levels of fluorescence detected for anti-6xHis-PE among cells incubated with ACDClxΔ15 when compared to control samples (FIGS. 4A and 4B). These results also show that CltxΔ15 is capable of selectively binding GBM cells without off-target toxicity when part of a BiTE-like fusion protein The scFVs from an antibody against CD3c was also shown to bind to T cells. Thus, the therapeutic fusion protein described herein is able to bring CD8+ T cells to glioblastoma cells as shown in FIG. 1B. Accordingly, a method of treating as subject having glioma and a method of producing a glioma-targeting therapeutic fusion protein are also described.

In some aspects, the method of treating a subject having glioma comprises administering to the subject a glioma-targeting therapeutic fusion protein, wherein the glioma-targeting therapeutic fusion protein comprises a truncated form of chlorotoxin that comprises a sequence with at least 75% homology to SEQ ID NO:1 linked to an anti-glioma therapeutic agent. The truncated form of chlorotoxin that may be linked to an anti-glioma therapeutic agent via an amino acid linker, for example, an amino acid linker consisting of at least one glycine residue and at least one serine residue. In some aspects, the truncated form of chlorotoxin is 13-18 amino acid residues in length. In particular implementations, the truncated form of chlorotoxin consists of the sequence set forth in SEQ ID NO:1. In some aspects, the anti-glioma therapeutic agent comprises a V_{H} domain and a V_{L} domain of an antibody against CD3. In particular implementations, the V_{H} domain of the antibody against CD3 comprises the sequence set forth in SEQ ID NO. 2 and the V_{L} domain of the antibody against CD3 comprises the sequence set forth in SEQ ID NO. 3. In such embodiments, a first amino acid linker sequence links the C-terminus of the V_{H} domain the antibody against CD3 to the N-terminus of V_{L} domains of the antibody against CD3 and a second amino acid linker sequence links the C-terminus of the truncated form of chlorotoxin to the N-terminus of the V_{H} domain of an antibody against CD3. In certain embodiments, the method of treating a subject having glioma comprises administering to the subject a glioma-targeting therapeutic fusion protein having the sequence set forth in SEQ ID NO:4 or SEQ ID NO:5.

The method of producing a glioma-targeting therapeutic fusion protein comprises providing a polypeptide of 13-18 amino acids residues comprising a sequence with at least 75% identity to SEQ ID NO:1; providing an anti-glioma therapeutic agent; and linking the polypeptide to the anti-glioma therapeutic agent to produce the glioma-targeting therapeutic fusion protein. In some embodiments, the C-terminus of the polypeptide is linked to the anti-glioma therapeutic agent. In certain implementations, the step of linking anti-glioma therapeutic agent to the polypeptide comprises linking the anti-glioma therapeutic agent and the polypeptide with an amino acid linker sequence. In some aspects, the amino acid linker sequence consists of at least one glycine residue and at least one serine residue. In certain embodiments, the anti-glioma therapeutic agent comprises a V_{H} domain and a V_{L} domain of an antibody against CD3. For example, the amino acid sequence of the glioma-targeting therapeutic fusion protein comprises the sequence set forth in SEQ ID NO:4 or SEQ ID NO:5.

An expression plasmid for expressing the therapeutic peptide or the glioma-targeting therapeutic fusion protein is also described. The nucleotide sequence of the expression plasmid is set forth in SEQ ID NO:6. In some embodiments, the nucleotide sequence of the expression plasmid is a sequence having at least 70% identity, at least 80% identity, at least 90% identity, or at least 95% identity to SEQ ID NO:6.

### Examples

The present invention is further illustrated by the following examples that should not be construed as limiting. The contents of all references, patents, and published patent applications cited throughout this application, as well as the Figures, are incorporated herein by reference in their entirety for all purposes.

### I. Design and Production of ACDClxΔ15

The amino acid sequence of ACDClxΔ15 was composed from previously published sequences of the VH and VL scFvs of the mouse anti-CD3 ε mAb 2C11 and a truncated chlorotoxin molecule which was obtained through deletion of 22 amino acids from the C-terminus of full-length chlorotoxin. These sequences were joined by a (Gly₄ Ser)₃ linker and an 8x histidine tag (His-tag) was included at the N-terminus.

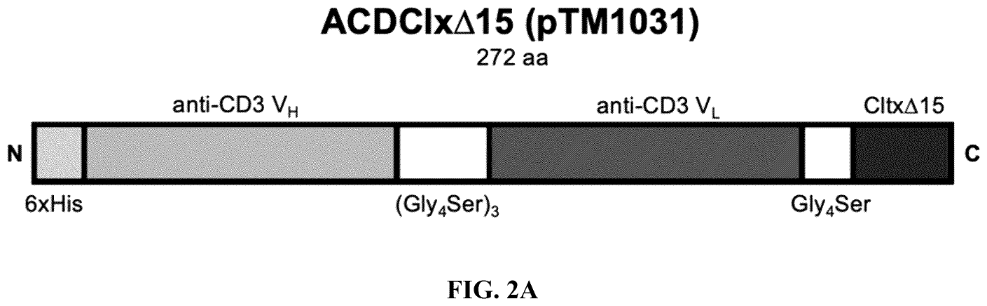

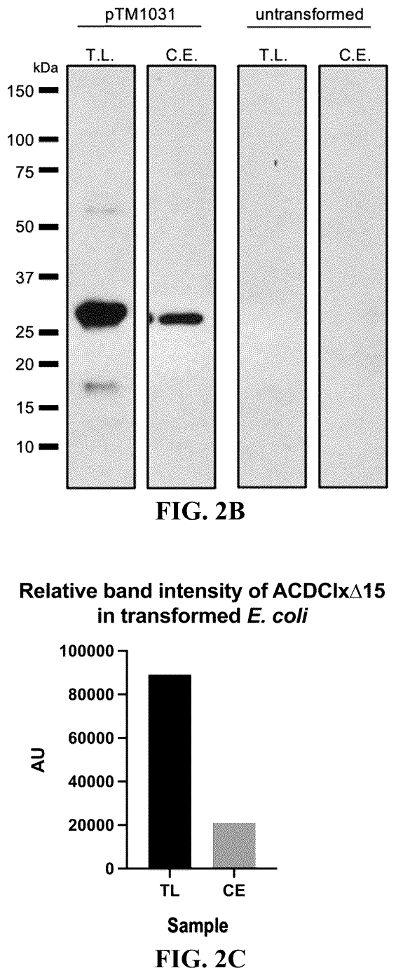

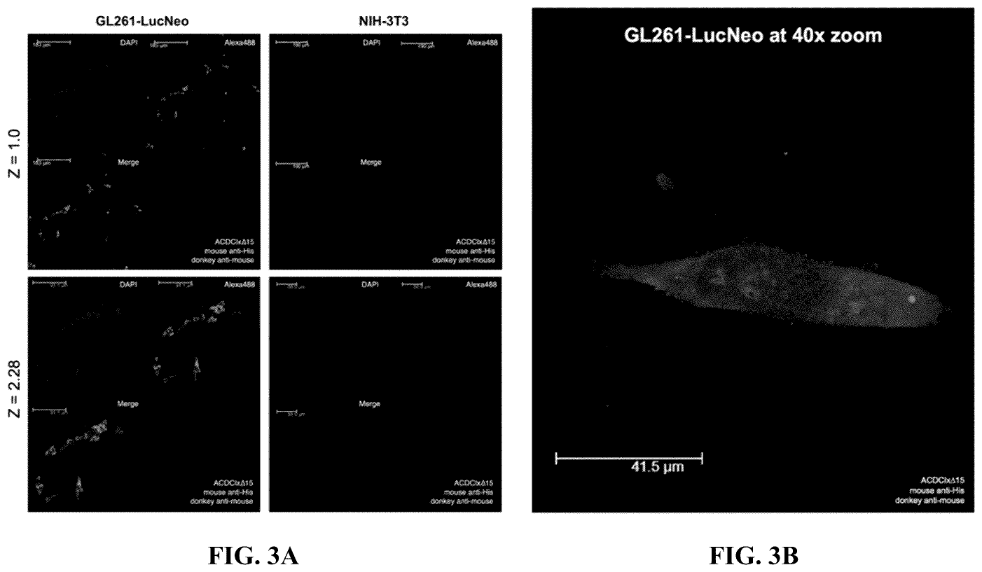

In order to produce ACDClxΔ15, the gene encoding its components was cloned into a plasmid capable of transforming electrocompetent E. coli cells (FIG. 2A). The gene encoding ACDClxΔ15 was cloned into an ampicillin resistant pET-11 a plasmid, and the resulting vector (pTM1031, nucleotide sequence set forth in SEQ ID NO:6) was used to transform electrocompetent BL21 (DE3) E. coli cells by electroporation.

Transformed cells recovered in SOC medium (CSH Protocols, 2006) for 1 hr at 37° C. before growing overnight on agar plates treated with ampicillin. Antibiotic selection and PCR screening confirmed cell colonies positive for pTM1031; positive colonies were then grown in 1 L cultures and induced with 0.3 mM IPTG after 4 hrs of shaking. Cultures were then centrifuged at 6000×g and 4° C. for 20 min (Beckman JA-17 rotor #369691). Pelleted cells were resuspended in extraction buffer (150 mM NaCl, 50 mM Tris pH 8, and 2 mM EDTA in PBS) and freeze-thawed twice to lyse cells. Resuspended cells were vortexed and incubated at 30° C. on a shaker before adding 0.5% PMSF and 20 mg lysozyme. In 45 min increments, the following were added: 0.5% Triton X-100, followed by 0.18% DNAse I and 1 M MgSO4. Samples receiving s-sulfonation to prevent DSB formation were denatured and s-sulfonated overnight at 4° C. in buffer containing 6 M guanidine, 200 mM sodium sulfite, 150 mM sodium tetrathionate dihydrate, 50 mM Tris pH 8, and 150 mM NaCl.

All samples were purified via metal-affinity chromatography by incubating overnight at 4° C. with Roche cOmplete™ nickel affinity resin. Purified protein was eluted through the addition of 1 M imidazole to the column. Concentration of the resulting eluent was measured on an ND-1000 NanoDrop spectrophotometer. S-sulfonated samples were then refolded by diluting 1:100 v/v overnight at 4° C. in oxidizing buffer (0.5 M arginine, 2 mM oxidized glutathione, 100 mM Tris base, and 2 mM EDTA in PBS). Samples were re-concentrated by centrifugation in Millipore Amicon Ultra-15 centrifugal filter tubes with a 3.5 kDa MW cutoff (GE Healthcare, Pittsburgh, PA) for 15 min at 4° C. in a Beckman-Coulter JS-5.3 swinging-bucket rotor before being exchanged into PBS on a Sephadex G-25 desalting column. Mock-purified protein refers to samples collected from BL21 (DE3) E. coli cells that were not transformed with the pTM1031 plasmid. These samples were treated identically to those produced from transformed cells as described above.

### II. SDS-PAGE and Immunoblotting

Following induction with IPTG, cells were harvested and ACDClxΔ15 was present in insoluble inclusion bodies within the total cell lysate of transformed cells but not in untransformed cells, as detected by Western blot (FIG. 2B).

To verify the presence and purity of ACDClxΔ15 following purification, samples were mixed with 6x Laemmli buffer (4% SDS, 20% glycerol, 0.004% bromophenol blue, 0.125 M Tris-HCl, and 10% DTT in ddH₂O), boiled for 5 min at 100° C. and briefly centrifuged before loading onto 4-20% Mini-PROTEAN TGX stain-free polyacrylamide gels (Bio-Rad) alongside Bio-Rad Precision Plus dual-color protein standards. Gels were run at 150 V for 40 min, or until the sample buffer reached the bottom of the gel.

Following SDS-PAGE, gels were imaged for total protein via UV transillumination before being transferred to nitrocellulose membranes on a Trans-Blot® SD semi-dry transfer cell at 15 V for 15 min. Membranes were blocked in PB ST+2.5% non-fat milk (PBST-M) solution for 1 hr at RT and washed with PBST for 15 min before staining with mouse anti-His primary Ab (Sigma H1029) for 30 min. Membranes were again washed with PBST for 15 mins before donkey anti-mouse HRP secondary Ab (Jackson ImmunoResearch #715-305-150) for 30 min. Following a final 15 min wash in PBST, membranes were incubated with HRP substrate for 1 min and exposed onto X-ray film for 30 sec in the dark.

Purification and subsequent concentration resulted in a 24% yield of ACDClxΔ15 (FIG. 2C). Any proteins present in untransformed samples (mock-purified protein) were not detected by anti-His antibody and therefore were not expected to be capable of binding nickel resin for purification.

### III. Immunocytochemistry

For the first test of CltxΔ15's ability to bind GBM cells as part of a BiTE-like molecule, ACDClxΔ15 was incubated with GL261-LucNeo mouse glioblastoma cells and stained with antibodies which fluoresce when excited with lasers at a wavelength of 488 nm. GL261-LucNeo mouse glioblastoma cells and NIH-3T3 mouse embryonic fibroblasts (ATCC), generously provided by Dr. Adrienne C. Scheck at Barrow Neurological Institute and Dr. Debra P. Baluch at Arizona State University, respectively, were grown in culture media (DMEM+10% FBS/F12+1% PSG) at 37° C. to approximately 70% confluency. Cells were passaged and split 1:6 before seeding on four coverslips per cell line in separate wells of two 6-well plates. Cells were grown to approximately 90% confluency on coverslips before fixing in 1 mL 2% paraformaldehyde. Coverslips were washed with 1 mL PBS+5% nonfat milk (PBS-M) before adding 1 mL of 1 μM ACDClxΔ15 in PBS for 1 hr on the rocker at 4° C. Cells were washed with 1 mL PBS-M 3x for 5 min each on a rocker at RT. Mouse anti-6xHis primary antibody was added to all coverslips except secondary control at a 1:500 dilution in 1 mL PBS-M and incubated on rocker at 4° C. overnight. Primary antibody was removed before adding 1 mL of secondary antibody (donkey anti-mouse IgG H&L Alexa Fluor® 488, Abcam ab150109) and incubating in the dark at 4° C. on a rocker for 1 hr. Secondary antibody was removed and coverslips were washed with PBS-M 3x for 5 min each on rocker. Antibody wash was removed, and cells were incubated with DAPI nuclear stain for 30 min before mounting and sealing coverslips on microscope slides. Slides were imaged on a Leica SP8 White Light Laser Confocal microscope using a 488 nm wavelength laser.

When incubated with ACDClxΔ15 and visualized with fluorescent confocal microscopy, GBM cells displayed bright green fluorescence compared to mouse embryonic fibroblast cells (NIH-3T3) also incubated with ACDClxΔ15 (FIGS. 3A and 3B). Control samples receiving either mock protein, primary and secondary Ab only, or ACDClxΔ15 and secondary Ab alone displayed negligible levels of fluorescence (FIGS. 3C and 3D). As a quantitative method to measure ACDClxΔ15-GBM binding, GL261-LucNeo cells were incubated with PBS, mock protein, anti-6xHis-PE, or ACDClxΔ15 and analyzed using flow cytometry. A distinct peak was observed for samples treated with ACDClxΔ15 but not those receiving controls (FIG. 4A). Dot plots of all events recorded per sample showed similar results, with clear increases in cells positive for anti-6xHis-PE observed compared to controls (FIG. 4B). After gating for positive cells based on these negative controls, the percent of positive cells was calculated and found to be higher in samples treated with ACDClxΔ15, with 35.5% of cells displaying fluorescence compared to 0.92%, 0.59%, and 0.59% for samples treated with PBS, anti-6xHis, and mock protein respectively (FIG. 4C). Median fluorescent intensity of positive events was likewise higher in ACDClxΔ15 samples than in controls (FIG. 4D). Together, these results indicate that ACDClxΔ15 is capable of binding murine GBM cells, but not other mouse tissue types, in vitro.

### IV. Analyzing GBM Binding Via Flow Cytometry

After testing its ability to bind mouse GBM cells specifically via fluorescence microscopy and flow cytometry, ACDClxΔ15 was then co-incubated with GBM cells and splenocytes to determine whether or not it was capable of both binding and activating T cells in the presence of GBM. To achieve this, flow cytometry was again utilized to measure fluorescent signals of co-incubated GBM and T cells treated with specific antibodies that would detect the presence of important markers of T cell binding and activation. Specifically, T cells were stained for the presence of CD4 and CD8 and incubated with GBM cells stained for beta tubulin and binding events were determined by the presence of GBM and either CD4 or CD8 probes in single recorded events.

GL261-LucNeo cells were grown in culture media (DMEM+10% FBS+1% pen/strep) to approximately 90% confluency. Cells were harvested and washed with FACS buffer (1% bovine serum albumin and 0.1% sodium azide in PBS) and aliquoted into a 96-well round bottom plate (approx. 1.0×10⁶ cells/well). Cells were washed 2× with FACS buffer by centrifugation for 5 min at 1200 rpm on a Thermoscientific Bioliner rotor (#75003667) before being incubated with either 5 μM ACDClxΔ15, mock-purified protein, or PBS for 30 min at 4° C. Anti-6xHis-PE antibody was added to all samples except one PBS-only control at a 1:200 dilution in FACS buffer and incubated for 30 min at 4° C. Samples were again washed as before and resuspended in 1 mL FACS buffer before running on a BD LSRFortessa™ flow cytometer. Results were analyzed using FlowJo™ v10.8 software (BD Life Sciences).

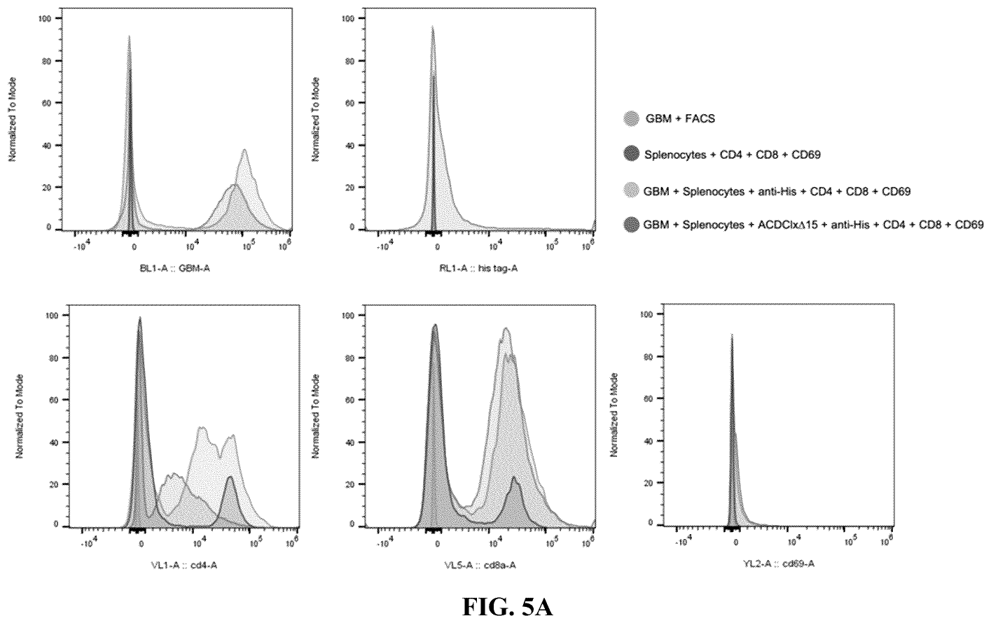

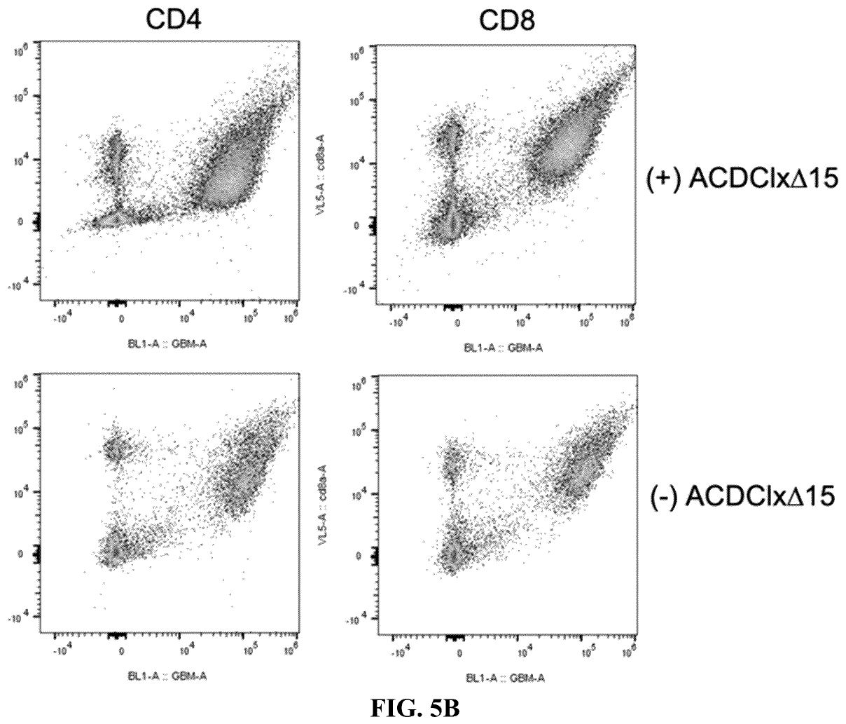

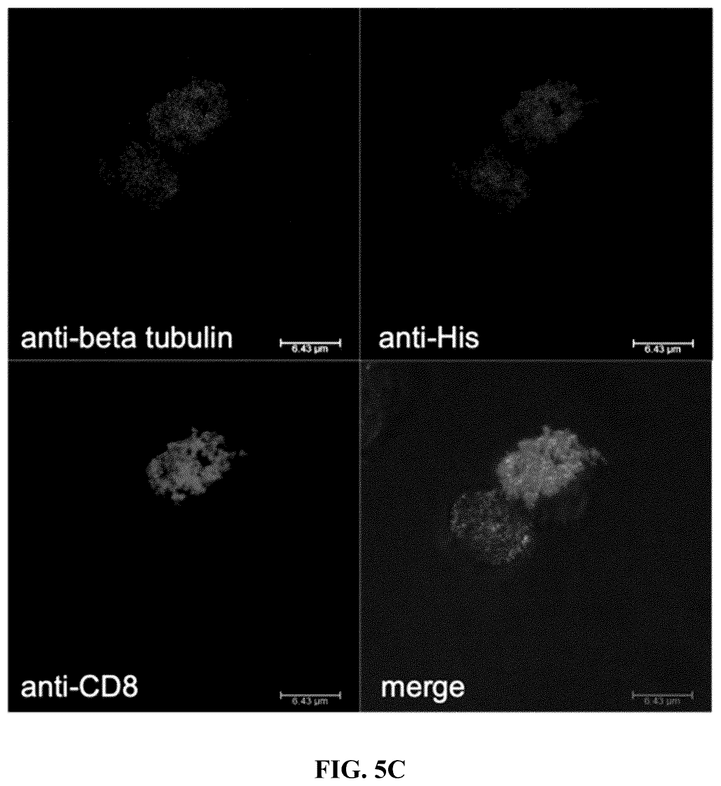

Samples of GBM and T cells co-incubated with or without ACDClxΔ15 displayed prominent peaks when probed with anti-beta tubulin, anti-CD4, and anti-CD8, which act as markers for these cells (FIG. 5A). The sample which received ACDClxΔ15 showed a small signal peak for its corresponding anti-His detection while all other samples displayed negligible fluorescence.

### V. Analyzing GBM-T Cell Interactions Via Flow Cytometry and FACS

Following flow cytometry, cells were sorted using FACS to isolate events that contained GBM cells, CD8+T cells, and ACDClxΔ15.

GL261-LucNeo cells were grown and prepared for flow cytometry as described in Example IV and aliquoted into 15 mL Falcon™ tubes. Spleens were harvested from wild-type mice and splenocytes were isolated by straining spleens into RPMI media. Red blood cells were lysed by centrifugation for 5 min at 1200 rpm and 4° C. before incubating in 1 mL Ack Lysis buffer for 2 min at RT. Cells were quenched with RPMI and centrifuged as above before resuspending in 8 mL final volume of RPMI on ice.

Splenocytes were counted and approximately 1.0×10⁶ cells were aliquoted into separate Falcon™ tubes and incubated with their respective treatments. Antibodies used in this experiment are listed in Table 2, and 1 mL ACDClxΔ15 was added to samples at 1 μM concentration. All incubations were performed for 30 min at 4° C. in the dark except for anti-CD69, which was incubated for 2 hrs. Two washes were performed between each incubation by centrifuging samples at 1200 rpm for 5 min at 4° C. and resuspending cells in 2 mL FACS buffer. Following the final wash, samples were resuspended in 1 mL FACS buffer and collected on a ThermoFischer Attune NxT flow cytometer before analyzing as described in Example IV.

TABLE 2
Fluorescent antibodies used for flow
cytometry and FACS experiments.

| Antibody | Fluorophore | Catalog # |
|---|---|---|
| anti-6xHis | APC | 362605 (BioLegend) |
| anti-beta tubulin | Alexa Fluor@488 | A4-251-C100 (EXBIO) |
| anti-CD4 | V450 | FP20224002 (MBL Internatioi |
| anti-CD8a | Brilliant Violet 711 | 100747 (BioLegend) |
| anti-CD69 | PE-Damie 594 | 104535 (BioLegend) |

Samples obtained were run on a BD FACSAria™ IIu cell sorter in order to identify samples positive for both GBM and T cells. Samples in which GBM and splenocytes were co-incubated with ACDClxΔ15 and all five antibodies were sorted for events that were positive for anti-6xHis and either anti-CD4 or anti-CD8 to indicate GBM-T cell binding events. Resulting samples were plated onto coverslips and fixed before imaging on a Lecia SP8 confocal microscope.

These samples were imaged using confocal microscopy in order to visualize binding events between GBM and T cells (FIG. 5C). Fluorescence was detected for all three probed antibodies, and a CD8+ cell was observed in close proximity to a cell positive for anti-beta tubulin, the marker used here to identify GBM cells. However, isolated scans show that this anti-beta tubulin signal was also present on the CD8+ cell.

### VI. Analysis of T Cell Activation by ACDClxΔ15

Calcium flux is an indicator of initial T cell activation. Calcium flux (as indicated by an increase in Fluo-4 AM fluorescence intensity using FITC excitation/emission filter) are measured immediately after mixing GL261-LucNeo mouse glioma cells with freshly isolated mouse splenocytes with (1) nothing added, (2) ACDClxΔ15 protein buffer (no ACDClxΔ15), (3) ACDClxΔ15 added, or (4) anti-CD3 antibody which acts as a positive control for T cell activation. Four equivalent samples are measured consecutively: Splenocytes+GL261 cells (nothing added); Splenocytes+GL261+protein buffer; Splenocytes+GL261+ACDClxΔ15, and Splenocytes+GL261+anti-CD3 antibody.

CD4+ and CD8+T cell activation are measured by CD69 expression 36 hours post-treatment. The treatment comprises: Media (negative control), 1 μM ACDClxΔ15, or anti-CD3 antibody (positive control). Treatments are incubated with freshly isolated mouse splenocytes (B6 background) and confluent GL261-LucNeo mouse GBM cells.

What is claimed is:

1. A polypeptide of 13-18 amino acids residues comprising a sequence with at least 75% identity to SEQ ID NO:1.

2. The polypeptide of claim 1, wherein the polypeptide comprises the sequence of SEQ ID NO:1.

3. The polypeptide of claim 1, further comprising a therapeutic agent against glioma, wherein the therapeutic agent against glioma is linked to the polypeptide and the polypeptide binds to glioma cells.

4. The polypeptide of claim 3, wherein the therapeutic agent against glioma is linked to the C-terminus of the polypeptide.

5. A therapeutic fusion protein comprising:
a truncated form of chlorotoxin being 13-18 amino acid residues comprising a sequence with at least 75% homology to SEQ ID NO:1;
a V_{H} domain of an antibody against CD3; and
a V_{L} domain of an antibody against CD3,
wherein a first amino acid linker sequence links the C-terminus of the V_{H} domain the antibody against CD3 to the N-terminus of V_{L} domains of the antibody against CD3 and a second amino acid linker sequence links the C-terminus of the truncated form of chlorotoxin to the N-terminus of the V_{H} domain of an antibody against CD3.

6. The therapeutic fusion protein of claim 5, wherein the truncated form of chlorotoxin comprises the sequence of SEQ ID NO:1.

7. The therapeutic fusion protein of claim 5, wherein the V_{H} domain of the antibody against CD3 comprises the sequence set forth in SEQ ID NO:2.

8. The therapeutic fusion protein of claim 5, wherein the V_{L} domain of the antibody against CD3 comprises the sequence set forth in SEQ ID NO:3.

9. The therapeutic fusion protein of claim 5, wherein the first amino acid linker and the second amino acid linkers consist of at least one glycine residue and at least one serine residue.

10. The therapeutic fusion protein of claim 5, wherein the amino acid sequence of the therapeutic fusion protein comprises the sequence set forth in SEQ ID NO:4 or SEQ ID NO:5.

11. A method of treating a subject having glioma, the method comprising:
administering to the subject a glioma-targeting therapeutic fusion protein, wherein the glioma-targeting therapeutic fusion protein comprises:
a truncated form of chlorotoxin being 13-18 amino acid residues comprising a sequence with at least 75% homology to SEQ ID NO:1, and
an anti-glioma therapeutic agent, wherein the anti-glioma therapeutic agent is linked to the truncated form of chlorotoxin.

12. The method of claim 11, wherein the truncated form of chlorotoxin consists of the sequence set forth in SEQ ID NO:1.

13. The method of claim 11, wherein the anti-glioma therapeutic agent comprises a V_{H} domain and a V_{L} domain of an antibody against CD3.

14. The method of claim 13, wherein the amino acid sequence of the glioma-targeting therapeutic fusion protein comprises the sequence set forth in SEQ ID NO:4 or SEQ ID NO:5.

15. A method of producing a glioma-targeting therapeutic fusion protein, the method comprising:
providing the polypeptide of claim 1;
providing an anti-glioma therapeutic agent; and
linking the polypeptide to the anti-glioma therapeutic agent to produce the glioma-targeting therapeutic fusion protein.

16. The method of claim 15, wherein the C-terminus of the polypeptide is linked to the anti-glioma therapeutic agent.

17. The method of claim 15, wherein the step of linking anti-glioma therapeutic agent to the polypeptide comprises linking the anti-glioma therapeutic agent and the polypeptide with an amino acid linker sequence.

18. The method of claim 15, wherein the anti-glioma therapeutic agent comprises a V_{H} domain and a V_{L} domain of an antibody against CD3.

19. The method of claim 15, wherein the amino acid sequence of the glioma-targeting therapeutic fusion protein comprises the sequence set forth in SEQ ID NO:4 or SEQ ID NO:5.

20. A plasmid for expressing the therapeutic fusion protein of claim 10, wherein the sequence of the plasmid comprises SEQ ID NO:6.
